(19) United States
(12) Patent Application Publication
(10) Pub. No.: US 2013/0165749 A1
(43) Pub. Date: Jun. 27, 2013

(54) ENDOSCOPE CAPABLE OF CAPTURING 3D IMAGE

(71) Applicants: Yi-Chen Chou, Tainan City (TW); Yung-Che Chung, Tainan City (TW)

(72) Inventors: Yi-Chen Chou, Tainan City (TW); Yung-Che Chung, Tainan City (TW)

(73) Assignee: HIMAX IMAGING LIMITED, Tainan City (TW)

(21) Appl. No.: 13/335,706

(22) Filed: Dec. 22, 2011

(51) Int. Cl. A61B 1/04

(52) U.S. Cl. 600/109

(57) ABSTRACT

An endoscope including a tube and a distal section is provided. The distal section, coupled to a distal end of the tube, includes a left wafer-level image sensor, disposed to capture at least one left image, and a right wafer-level image sensor, disposed to capture at least one right image. Wherein, the captured left image and right image are processed as a 3D image.

### BACKGROUND OF THE INVENTION

1. Field of the Invention

The present invention, generally relates to an endoscope, and more particularly to an endoscope capable of capturing a 3D image.

2. Description of Related Art

An endoscope is an instrument that is capable of being inserted into an organ to examine the interior of the organ. The endoscope generally includes a flexible tube and a lens system. disposed at a distal end of the endoscope for collecting images of the interior of the organ.

Due to the miniature dimension of the endoscope, the manufacturing of the lens system requires great effort and. thus making the overall cost high. Moreover, formerly the image collected by the lens system is two-dimensional (2D) image source, which lacks a sense of reality when observing. As far as the cost and practicality are concerned, since the conventional endoscope is not only high-priced but also unable to generate 3D image, a need. has arisen to propose a novel endoscope that eliminates the problems mentioned above.

### SUMMARY OF THE INVENTION

In view of the foregoing, the embodiment of the present invention provides an endoscope which has a distal section made of a wafer-level imaging module including a left and a right wafer-level image sensor. The endoscope of the embodiment may not only reduce the overall cost of the endoscope, but also obtain 3D images.

According to one embodiment, an endoscope including a tube and a distal section is provided. The distal section, coupled to a distal end of the tube, includes a left wafer-level image sensor, disposed to capture at least one left image, and a right wafer-level image sensor, disposed to capture at least one right image. Wherein, the captured left image and right image are processed as a 3D image.

### BRIEF DESCRIPTION OF THE DRAWINGS

FIG. 1. schematically shows a lateral cross-sectional view of an endoscope according to one embodiment of the present invention;

FIG. 2 schematically shows a perspective view of the distal section of FIG. 1 according to one embodiment of the present invention;

FIG. 3 schematically shows a. perspective view of the distal section of FIG. 1 according to another embodiment of the present invention; and

FIG. 4 schematically shows a perspective view of the distal section of FIG. 1 according to a further embodiment of the present invention.

### DETAILED DESCRIPTION OF THE INVENTION

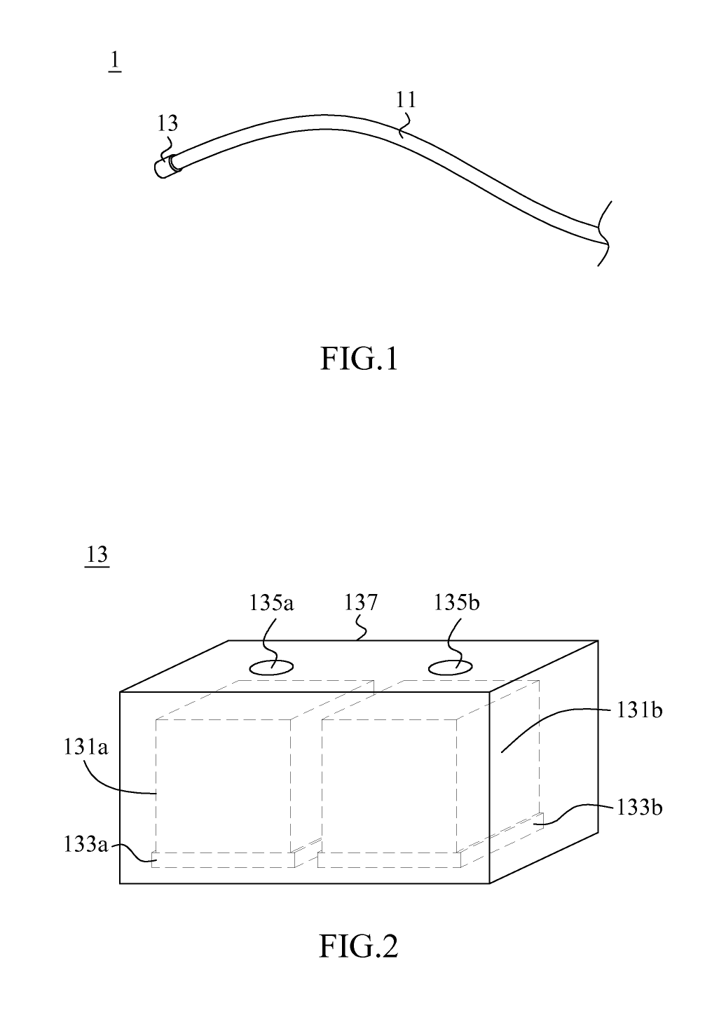

FIG. 1 schematically shows a lateral cross-sectional view of an endoscope 1 according to one embodiment of the present invention. As shown in FIG. 1, the endoscope 1 includes a tube 11 and a distal section 13. Specifically, the distal section 13 is disposed. at and coupled to a distal end of the tube 11. The distal section 13 of the embodiment primarily includes at least one wafer-level imaging module (or wafer-level module WLM, for short).

FIG. 2 schematically shows a perspective view of the distal section 13 according to one embodiment of the present invention. As shown in FIG. 2, the distal section 13 includes a left and a right wafer-level module. The left wafer-level module contains a left wafer-level image sensor 131a and a left wafer-level optics (WLO) 133a, while the right wafer-level module contains a right wafer-level image sensor 131b and a right wafer-level optics 133b. The left wafer-level image sensor 131a and the right wafer-level image sensor 131b are situated facing the distal end of the tube 11, and may be, but not limited to, a complementary metal oxide semiconductor (CMOS) image sensor (commonly abbreviated as CIS). The left wafer-level optics 133a and the right wafer-level optics 133b, such as a lens, is situated away from the distal end of the tube 11, and may be made of, but not limited, to, glass. The left wafer-level image sensor 131a, the right wafer-level image sensor 131b and the left wafer-level optics 133a, the right wafer-level optics 133b may be bonded together, respectively, for example, with an adhesive. Compared to the conventional endoscope, the endoscope of the present embodiment makes use of the mass-productivity and low cost of semiconductor technique to manufacture the imaging system of the endoscope. Wafer-level module is a technique of fabricating miniaturized optics such as lens module or camera module at the wafer level using semiconductor techniques, and details of manufacturing the wafer-level imaging module 12 may be referred, for example, to U.S. Pat. No. 7,564,496 to Wolterink et al., entitled “Camera device, method of manufacturing a camera device, wafer scale package,” the disclosure of which is incorporated herein, by reference.

The distal section 13 further includes a holder 137 for housing the left wafer-level module, containing the left wafer-level image sensor 131a and the left wafer-level optics 133a, and the right wafer-level module, containing the right wafer-level image sensor 131b and the right wafer-level optics 133b. The left wafer-level module is aligned with the right wafer-level module in the holder 137. In one embodiment, the holder 137 has two openings 135a, 135b situated above and aligned with the left and right wafer-level modules. So that the left wafer-level image sensor 131a may capture at least one left image via the openings 135a, and the right wafer-level image sensor 131b may capture at least one right image via the openings 135b, and then the captured left and right images may be processed as a 3D image.

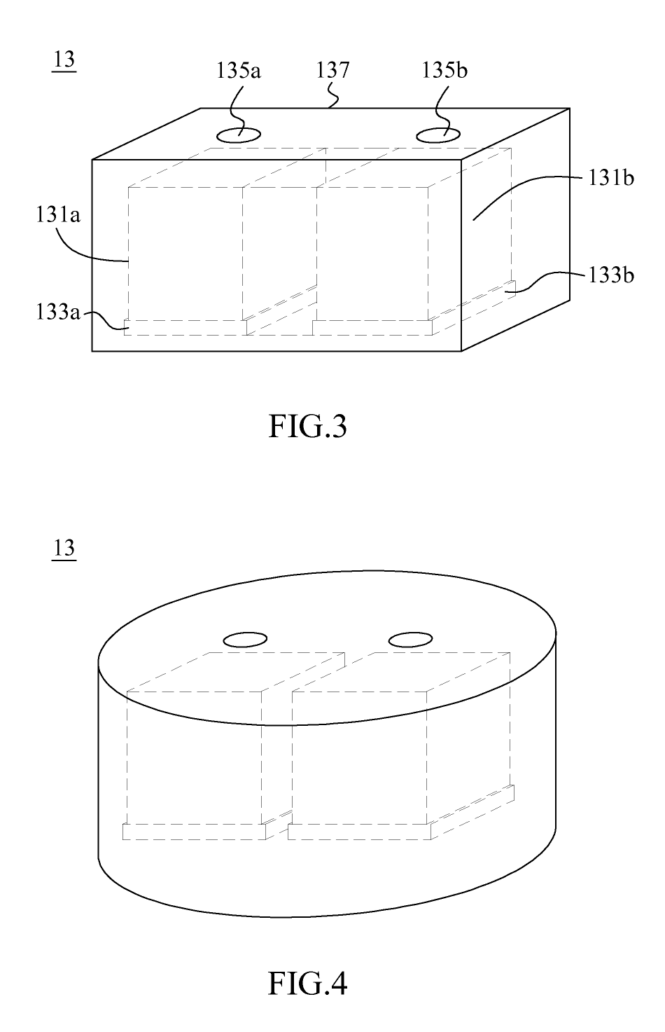

Afterwards, referring to FIG. 3, it schematically shows a perspective view of the distal section 13 according to another embodiment of the present invention. Besides disposing two wafer-level modules, the left wafer-level image sensor 131a, the right wafer-level image sensor 131b, the left wafer-level optics 133a, and the right wafer-level optics 133b can be integrated in a single wafer-level module which is housed by the holder 137, as shown.

Finally, FIG. 4 schematically shows a perspective view of the distal section 13 according to a further embodiment of the present invention. Besides cubic shape, the holder 137 can be into the shape of a cylinder, as shown, so as to fit in with the tube 11.

According to the above embodiment, the endoscope, provided. in the present invention, integrates two wafer-level modules, so as to process the captured left and right images as a 3D image for observation or examination.

Although specific embodiments have been illustrated and described, it will be appreciated by those skilled in the art that various modifications may be made without departing from the scope of the present invention, which is intended to he limited. solely by the appended claims.

What is claimed is:

1. An endoscope, comprising:
a tube; and
a distal section coupled to a distal end of the tube, comprising:
a left wafer-level image sensor disposed to capture at least one left image; and
a right wafer-level image sensor disposed to capture at least one right image;
wherein, the captured left image and right image are processed as a 3D image.

2. The endoscope of claim 1, wherein the distal section further comprises:
a left wafer-level optics bonded with the left wafer-level image sensor; and
a right wafer-level optics bonded with the right wafer-level image sensor;
wherein, the left and right wafer-level image sensors are situated facing the distal end of the tube, and the left and right wafer-level optics are situated away from the distal end of the tube.

3. The endoscope of claim 2, further comprising:
a holder disposed to house the distal section.

4. The endoscope of claim 3, wherein, the left wafer-level image sensor, the right wafer-level image sensor, the left wafer-level optics and the right wafer-level optics are contained by a single wafer-level module.

5. The endoscope of claim 4, wherein the holder houses the single wafer-level module.

6. The endoscope of claim 3, wherein, the left wafer-level image sensor and the left wafer-level optics are contained by a left wafer-level module, and the right wafer-level image sensor and the right wafer-level optics are contained, by a right wafer-level module.

7. The endoscope of claim 6, wherein the holder louses the left and right wafer-level modules, and the left wafer-level module is aligned. with the right wafer-level module.

8. The endoscope of claim 1, wherein the left and right wafer-level image sensors comprise a complementary metal oxide semiconductor (CMOS) image sensor.

9. The endoscope of claim 2, wherein the left and right wafer-level optics comprise a lens.

10. The endoscope of claim 9, wherein the lens is made of glass.

11. The endoscope of claim 5, wherein the holder has an opening situated above the single wafer-level module.

12. The endoscope of claim 7, wherein the holder has at least one opening situated above the left and right wafer-level modules.

13. The endoscope of claim 3, wherein the holder is into the shape of a cubic shape or a cylinder.
